(19) United States
(12) Patent Application Publication
(10) Pub. No.: US 2012/0148488 A1
(43) Pub. Date: Jun. 14, 2012

(54) Targeting Kidney Mesangium With Nanoparticles of Defined Diameter

(75) Inventors: Mark E. Davis, Pasadena, CA (US); Chung Hang J. Choi, (US); Jonathan Zuckerman, Valley Village, CA (US)

(73) Assignee: CALIFORNIA INSTITUTE OF TECHNOLOGY, Pasadena, CA (US)

(21) Appl. No.: 13/316,366

(22) Filed: Dec. 9, 2011

Related U.S. Application Data
(60) Provisional application No. 61/421,869, filed on Dec. 10, 2010.

(51) Int. Cl. A61K 31/7088; A61K 51/02; A61K 9/00; A61P 13/12; G01N 21/64; A61K 38/02; C12Q 1/02; B82Y 15/00; B82Y 5/00

(52) U.S. Cl. 424/1.11; 514/44 A; 514/1.1; 424/400; 435/29; 514/15.4; 977/773; 977/810; 977/915; 977/904

(57) ABSTRACT

Described herein are methods of treating a disorder affecting the mesangial cells in a subject by administering an engineered nanoparticle (ENP) capable of delivering a therapeutic agent to the subject. Also provided are diagnostic methods for administering to a subject an ENP, analyzing a mesangial cell of the subject and determining whether the engineered nanoparticle is present in a mesangial cell of the subject.

### CROSS REFERENCE TO RELATED APPLICATIONS

This application claims priority under 35 U.S.C. §119(e) to U.S. Provisional Application No. 61/421,869, filed on Dec. 10, 2010, which is incorporated herein by reference in its entirety.

This invention was made with government support under CA119347 awarded by the National Institutes of Health. The government has certain rights in the invention.

### FIELD OF THE INVENTION

The subject matter provided herein relates to nanoparticles capable of delivering therapeutic agents to mesangial cells of the kidney.

### BACKGROUND

Nanoparticles have demonstrated enormous potential for numerous medical applications, especially as an emerging class of carriers for drug delivery. To overcome biological barriers and reach their designated cellular destinations in sufficient amounts after their administration, nanoparticles often possess certain engineered physicochemical properties (e.g. size, charge, shape, and density of targeting ligands). Targeted nanoparticle delivery of therapeutic molecules has the potential to provide safer and more effective therapies for cancer applications. Recent work has focused on understanding the parameters that influence targeted nanoparticle behavior and the development of design rules for creating nanoparticle-based therapeutics and imaging agents. Passive tumor targeting takes advantage of the irregularity and leakiness of tumor vasculature to allow nanoparticle accumulation in the tumor (caused by the enhanced permeability and retention effect). Active targeting exploits the (over)expression of surface receptors on cancer cells by providing targeting ligands that can engage these receptors. (Choi, et al., PNAS 107(3):1235-40 (2010)). Despite proven success in particle accumulation at cellular locations and occurrence of downstream therapeutic effects (e.g. target gene inhibition) in a selected few organs such as tumor and liver, reports on effective delivery of engineered nanoparticles to other organs still remain scarce (Davis, et al., Mol. Pharm. 6, 659-668 (2009). Thus, additional efforts are needed to design nanoparticles that can reach other organs of the body as intact particles capable of delivering a therapeutic agent.

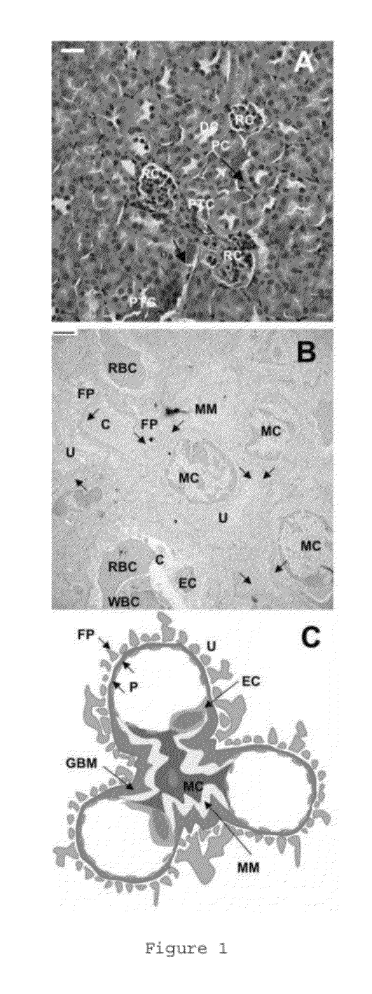

Constructing nanoparticles for drug delivery requires knowledge in colloidal science and biology, where biological constraints generally dictate the design of nanoparticle therapeutics and imaging agents. A celebrated design criterion is the notion of “renal clearance.” (Choi, et al. Nat. Biotechnol. 25, 1165-1170 (2007)). That is, nanoparticles will experience rapid clearance by the kidney if they are smaller than 10 nm in diameter. Such clearance originates from the innate function of the kidney as a blood filter. The structural and functional unit of the kidney—the nephron—consists of the renal corpuscle and tubule system. The renal corpuscle contains a tuft of blood capillaries and support tissue (the mesangium—see FIG. 1) called the glomerulus. A fraction of blood plasma entering the glomerulus will pass through the “glomerular filtration apparatus” to produce an ultrafiltrate, which will be collected by the tubule system and ultimately be processed into urine. The first component of the glomerular filtration apparatus is the glomerular endothelium with fenestrations that have been reported to be in the range of 80-100 nm in diameter. (Luft, F. C. et al. Antimicrob. Agents Chemother. 21, 830-835 (1982)). Next, the glomerular basement membrane (GBM), a 300-350 nm thick basal lamina rich in heparin sulfate and charged proteoglycans with an average pore size of 3 nm, filters small molecules by size and charge. Behind the GBM lies podocytes, cells with interdigitating foot processes that form “filtration slits” 32 nm wide. The glomerular filtration apparatus, taken in its entirety, possesses an effective size cutoff of 10 nm, and is responsible for the rapid “renal clearance” of small nanoparticles. Many nanoparticle-based contrasting agents for in vivo imaging were designed to be smaller than this size cutoff.

Closer examination of the renal corpuscle reveals the existence of another intriguing size cutoff that would affect the distribution pattern. Within the renal corpuscle, in the absence of GBM and podocytes, the sole dividing barrier between the mesangium (comprising mesangial cells and extracellular matrix) and the glomerular capillary lumen is the fenestrated endothelium. Sub-micron sized particles may feasibly diffuse and accumulate indefinitely in the mesangium once they depart from the capillary through these fenestrations. Such particles could serve as a useful means to target and deliver therapeutic agents to mesangial cells.

### SUMMARY

Described herein is a method of treating a disorder affecting the mesangial cells in a subject by administering an engineered nanoparticle (ENP) capable of delivering a therapeutic agent to the subject. There are a number of disorders that may affect the mesangial cells or physiological functions of the kidney reliant on proper mesangial cell function. Examples of such disorders include IgA nephropathy, lupus nephritis, diabetic nephropathy, focal segmental glomeruluosclerosis, membranous nephropathy, membranoproliferative glomerulonephritis, or amyloidosis. Other disorders affecting mesangial cell function are known to those skilled in the art. The methods provided herein require delivery of the described ENPs to a subject in need of treatment for a disorder affecting mesangial cells. For example, in some embodiments the described methods require systemic administration of an ENP. Once administered, the ENPs disclosed herein can produce a therapeutic effect in a variety of ways. For example, the ENP may carry a therapeutic agent to the kidney, enter a mesangial cell and initiate a therapeutic effect from inside the mesangial cell. In some embodiments the ENP may contact a mesangial cell of a subject as a fully intact particle. Further, in other embodiments the ENP may enter a mesangial cell of a subject as a fully intact particle. Subjects treatable by the described methods include mammals, such as a mouse, rat, hamster, rabbit, cat, dog, monkey, or chimpanzee. In some embodiments the subject treated by the described methods is a human.

The summary, as well as the following detailed description, is further understood when read in conjunction with the appended drawings.

FIG. 1 Illustrates the renal corpuscle and the mesangium. (a) Histology reveals the typical morphology of the renal cortex. Scale bar=10 μm. (b) This transmission electron micrograph shows the internal structures of the renal corpuscles. Scale bar=10 μm. (c) This schematic diagram shows the relationship between glomerular mesangial cells and glomerular capillaries (modified from Sakai and Kriz, Anat. Embryol. 176, 373-86 (1987)). Legend: renal corpuscles (RC), distal convoluted tubules (DC), proximal convoluted tubules (PC), peritubular capillaries (PTC), red blood cells (RBC), leukocytes (WBC), mesangial matrix (MM), mesangial cells (MC), foot processes of podocytes (FP), urinary space (U), glomerular capillary space (C), glomerular basement membrane (GBM), and pores (P) of the fenestrated endothelium of glomerular capillaries.

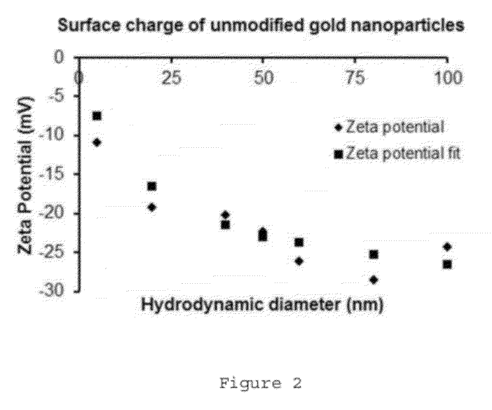

FIG. 2 Provides a graphical representation of dynamic light scattering (DLS) measurements of potential of unmodified gold nanoparticle matches reasonably with estimates based on Debye-Huckel electrokinetic theory.

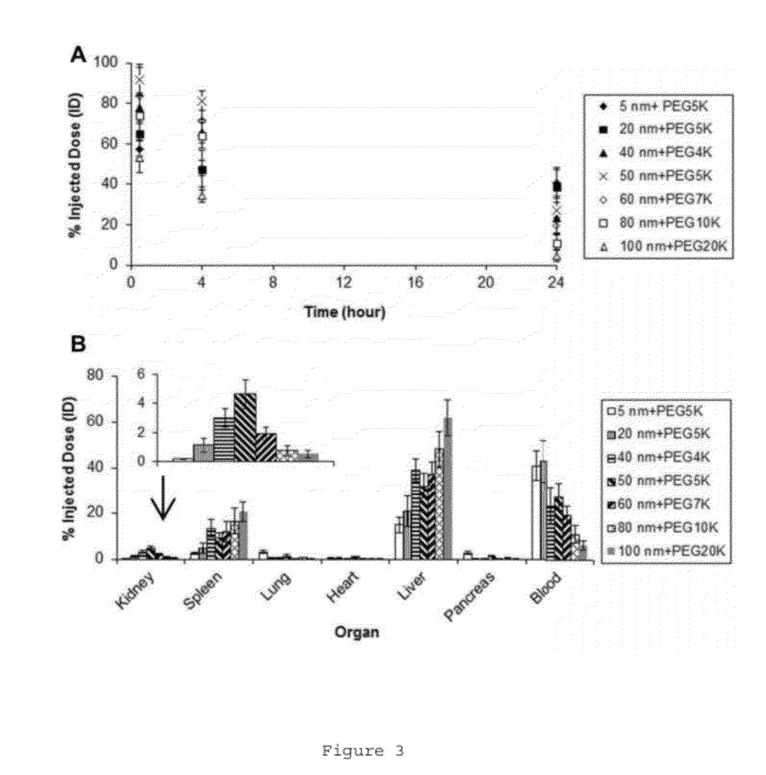

FIG. 3 Provides graphical illustrations of blood pharmacokinetics (a) and organ distribution for Auₓ-PEG_{y} NPs of various size. (a) Blood pharmacokinetics. All Auₓ-PEG_{y} NPs demonstrated superior colloidal stability and enhanced circulation in blood. (B) Organ level biodistribution. Bulk particle localization in the liver, spleen, and kidney was size dependent. Gold contents are normalized to % injected dose (% ID). Error bars indicate one standard deviation from each Auₓ-PEG_{y} NP class (N=3).

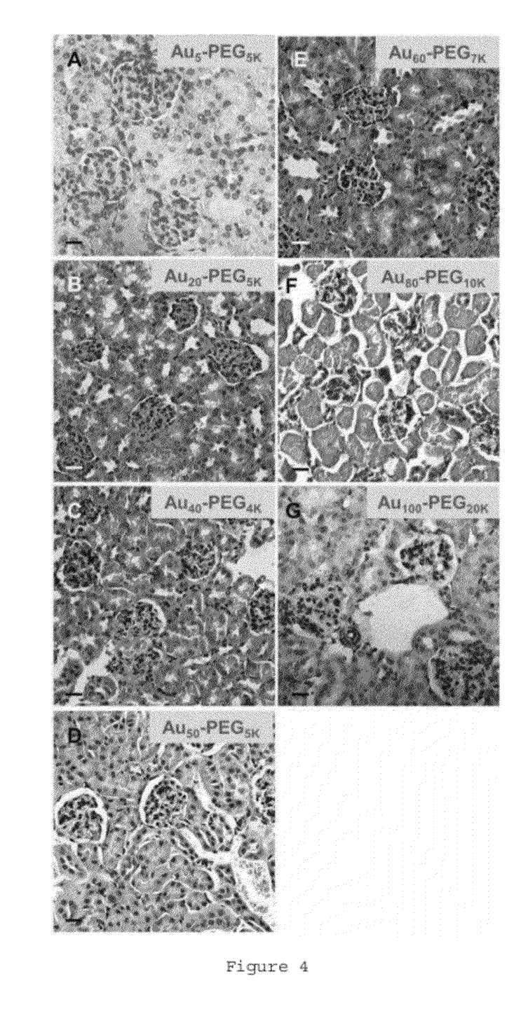

FIG. 4 Depicts light micrographs of “silver-enhanced” kidney sections that demonstrate the extent of glomerular targeting by particles. Auₓ-PEG_{y} NPs accumulated in a size-dependent manner, with Au₅₀-PEG₅₀₀₀ NPs displaying perfect glomerular targeting. Scale bar=10 μm.

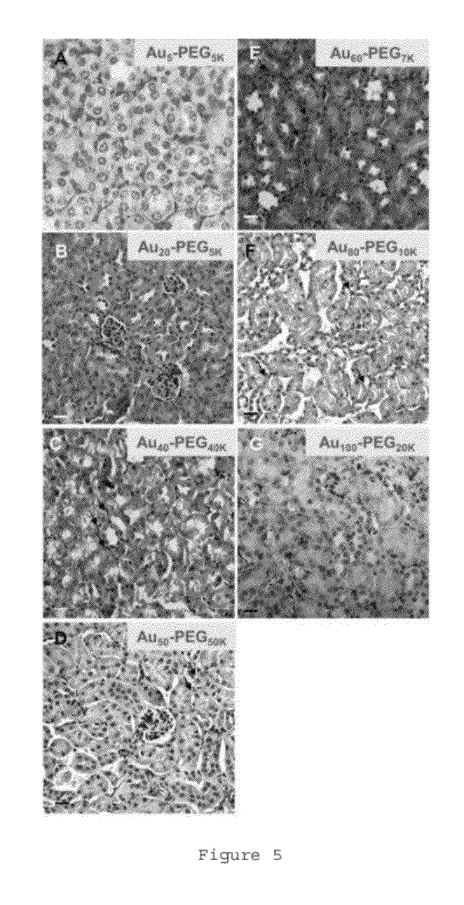

FIG. 5 Shows tissue-level accumulation of PEG-AuNPs of different sizes (A-G) in peritubular capillaries. The deposition of PEGylated gold nanoparticles in the renal cortex excluding renal corpuscles is not a function of particle size. Typically, particles are located adjacent to peritubular capillaries or in the connective tissue space between adjacent convoluted tubule cells. (Scale bar: 10 μm.)

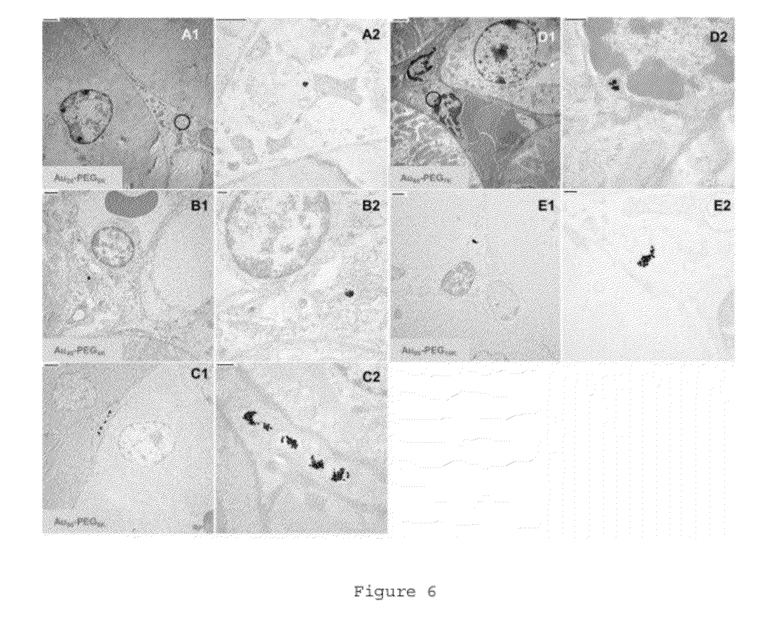

FIG. 6 Depicts cellular-level accumulation of PEG-AuNPs of different sizes (A-E) in peritubular capillaries. The deposition of Aux-PEGy NPs in the renal cortex excluding renal corpuscles is not a function of particle size. Particles are located adjacent to peritubular capillaries or in the connective tissue space between convoluted tubule cells. Images shown on the right are magnified versions of those shown on the left. (Scale bar: Left, 2 μm; Right, 500 nm.)

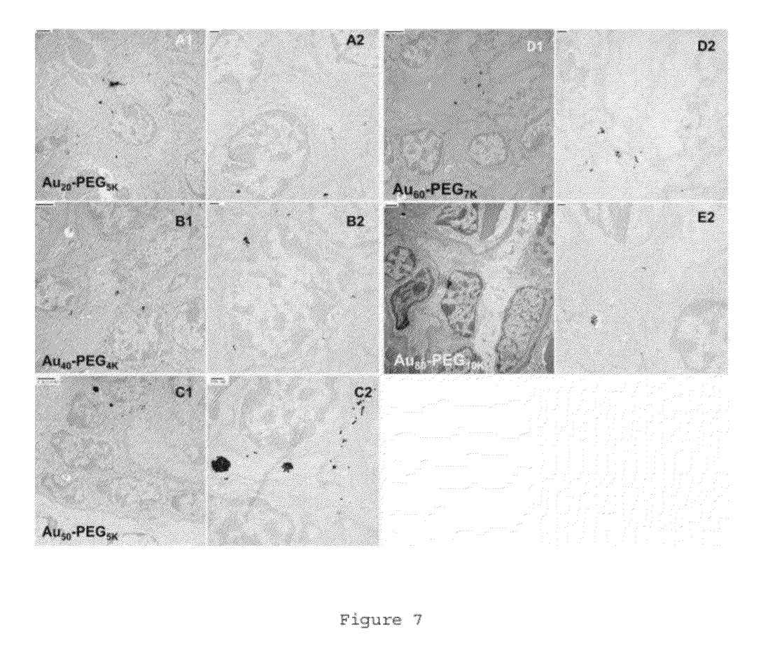

FIG. 7 Provides transmission electron micrographs of particle accumulation in the mesangium (mesangial cells and extracellular matrix). Images shown in the 2^{nd} column (scale bar=500 nm) are magnified versions of those in the 1^{st} column (scale bar=2 μm). Within renal corpuscles, Au₄₀-PEG₄₀₀₀, Au₅₀-PEG₅₀₀₀, and Au₆₀-PEG₇₀₀₀ NPs experienced most prominent uptake by mesangial cells. This indicates a range of particle size that leads to maximal association with mesangial cells.

### DETAILED DESCRIPTION OF ILLUSTRATIVE EMBODIMENTS

Described herein are methods of treating a disorder affecting the mesangial cells in a subject by administering an engineered nanoparticle (ENP) capable of delivering a therapeutic agent to the subject. Also provided are diagnostic methods for administering to a subject an ENP, analyzing a mesangial cell of the subject and determining whether the engineered nanoparticle is present in a mesangial cell of the subject. There are a number of disorders that may affect the mesangial cells or physiological functions of the kidney reliant on proper mesangial cell function. Examples of such disorders include IgA nephropathy, lupus nephritis, diabetic nephropathy, focal segmental glomeruluosclerosis, membranous nephropathy, membranoproliferative glomerulonephritis, or amyloidosis. Other disorders affecting mesangial cell function and targeted treatment efforts are known, or have been reported by, those skilled in the art. (See Tuffin et al., J. Am. Soc. Nephrol. 16:3295 (2005)). The described methods may be used to treat or identify subjects having such disorders.

### DEFINITIONS

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The term “biocompatible polymer” is used in a manner consistent with its meaning in the art. For example, biocompatible polymers include polymers that are neither themselves toxic to a subject (e.g., a mammal or human), nor degrade (if the polymer degrades) at a rate that produces monomeric or oligomeric subunits or other byproducts at toxic concentrations in the host. In certain embodiments of the present invention, biodegradation generally involves degradation of the polymer in an organism, e.g., into its monomeric subunits, which may be known to be effectively non-toxic. It is not necessary that any subject composition have a purity of 100% to be deemed biocompatible. Hence, a subject composition may comprise 99%, 98%, 97%, 96%, 95%, 90% 85%, 80%, 75% or even less of biocompatible polymers, e.g., including polymers and other materials and excipients described herein, and still be biocompatible.

The term “biologically active” as used herein refers to a compound which has an effect on a specific biological process. A biologically active compound has activity described at a molecular level such as receptor binding or blocking, receptor activation/inhibition, ion channel modulation, control of gene expression, by for example inhibitory RNA, and second messenger modulation. Biological activity further includes activity described at a cellular or subcellular level such as stimulation/inhibition of synaptic release. In addition, biological activity further includes activity described at an organismal level such as perceived relief of a symptom or increased organismal activity. In one embodiment, biological activity of a therapeutic compound includes inhibition of growth of a target cell, inhibition of division of a target cell and/or induction or stimulation of death of a target cell, such as a tumor cell. Biological activity of a compound is measurable and may be assessed by techniques known in the art.

The term “engineered nanoparticle” are non-naturally occurring nanoparticles that are synthesized or manufactured.

As used herein the term “subject” means a mammal, such as a rodent (mouse, rat, etc.), a rabbit, cat, dog, primate (monkey, ape, etc.), or a human.

The terms “targeting moiety” and “targeting agent” are used interchangeably and are intended to mean any agent, such as a functional group, that serves to target or direct the carrier particle to a particular location or association (e.g., a specific binding event). Thus, for example, a targeting moiety may be used to target a molecule to a specific target protein or enzyme, or to a particular cellular location, or to a particular cell type, to selectively enhance accumulation of the carrier particles. Suitable targeting moieties include, but are not limited to, polypeptides, nucleic acids, carbohydrates, lipids, hormones including proteinaceous and steroid hormones, growth factors, receptor ligands, antigens and antibodies, and the like.

As used herein the term “treatment” means administration to a subject one or more of the compositions provided herein. Depending on the context of the term, it may also mean executing the steps described for a method of treatment described herein. If treatment is administered, or carried out, prior to clinical manifestation of the unwanted condition (e.g., disease or other unwanted state of the host animal) then the treatment is prophylactic, i.e., it protects the host against developing the unwanted condition, whereas if it is administered, or carried out, after manifestation of the unwanted condition, the treatment is therapeutic, (i.e., it is intended to diminish, ameliorate, or stabilize the existing unwanted condition or associated side effects.)

As used herein, the term “therapeutic agent” includes any synthetic or naturally occurring biologically active compound which, when administered to a subject (human or nonhuman animal), induces a desired pharmacologic, immunogenic, and/or physiologic effect by local and/or systemic action. The term therefore encompasses those compounds or chemicals traditionally regarded as pharmaceutical agents, such as drugs, vaccines, and biopharmaceuticals including molecules such as proteins, peptides, hormones, nucleic acids, gene constructs and the like. More particularly, the term “therapeutic agent” includes compounds or compositions for use in all of the major therapeutic areas including, but not limited to, adjuvants; anti-infectives such as antibiotics and antiviral agents; analgesics and analgesic combinations, anorexics, antiinflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and polynucleotide molecules (polymeric forms of two or more nucleotides, either ribonucleotides (RNA) or deoxyribonucleotides (DNA) including both double- and single-stranded molecules, inhibitory RNA sequences, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

A “therapeutically effective amount” of a compound, with respect to a method of treatment, refers to an amount of the compound(s) in a preparation which, when administered to a subject as part of a desired dosage alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

### Compositions and Related Methods of Use

The methods provided herein require delivery of the described ENPs to a subject in need of treatment for a disorder affecting mesangial cells. For example, in some embodiments the described methods require systemic administration of an ENP. Administration may be carried out parenterally including but not limited to: systemic, subcutaneous, intravenous, intramuscular, intra-articular, intra-synovial, intrasternal, intranasal, topically, intrathecal, intrahepatic, intralesional, and intracranial injection or infusion techniques. Alternatively, the ENPs described herein may be administered intravenously or intraperitoneally, for example, by injection. The ENPs may be administered in a therapeutically effective amount, which can vary depending on the nature, or severity, of the disorder being treated. Once administered, the ENPs disclosed herein can produce a therapeutic effect in a variety of ways. For example, the ENP may carry a therapeutic agent to the kidney, enter a mesangial cell and initiate a therapeutic effect from inside the mesangial cell. In some embodiments the ENP may contact a mesangial cell of a subject as a fully intact particle. Further, in other embodiments the ENP may enter a mesangial cell of a subject as a fully intact particle. Subjects treatable by the described methods include mammals, such as a mouse, rat, hamster, rabbit, cat, dog, monkey, or chimpanzee. In some embodiments the subject treated by the described methods is a human.

The engineered nanoparticles associated with this method and provided herein may be synthesized in a variety of ways. For example a variety of materials may be used to produce the described ENPs. In some embodiments the ENPs may have a core, while in other embodiments they may not have a core. In the instance the ENP is designed to have a core, it may be made, wholly or in-part, from materials such as, but not limited to, gold, iron(III) oxide, carbon, carbon nanotubes, cadmium selenide, titanium, titanium dioxide, tin, tin oxide, silicon, silicon dioxide, iron, nickel, silver, copper, aluminum, steel, titanium alloy, brushite, tricalcium phosphate, chitosan, alumina, silica, lipinds, polystyrene, polylactides, silicone rubber, polycarbonate, polyurethane, polypropylene, polymethylmethaacrylate, polyvinyl chloride, polyester, polyether, or polyethylene. In some embodiments the core is composed of gold. The diameter of the core particle of an ENP may be from about 10 nm to about 100 nm. In some embodiments the diameter of the core may be from about 40 nm to about 75 nm. While in other embodiments the core diameter may be may be from about 50 nm to about 60 nm. In some embodiments the diameter of the core may be about 10 nm. In some embodiments the diameter of the core may be about 15 nm. In some embodiments the diameter of the core may be about 20 nm. In some embodiments the diameter of the core may be about 25 nm. In some embodiments the diameter of the core may be about 30 nm. In some embodiments the diameter of the core may be about 35 nm. In some embodiments the diameter of the core may be about 40 nm. In some embodiments the diameter of the core may be about 45 nm. In some embodiments the diameter of the core may be about 50 nm. In some embodiments the diameter of the core may be about 55 nm. In some embodiments the diameter of the core may be about 60 nm. In some embodiments the diameter of the core may be about 65 nm. In some embodiments the diameter of the core may be about 70 nm. In some embodiments the diameter of the core may be about 75 nm. In some embodiments the diameter of the core may be about 80 nm. In some embodiments the diameter of the core may be about 85 nm. In some embodiments the diameter of the core may be about 90 nm. In some embodiments the diameter of the core may be about 95 nm. In some embodiments the diameter of the core may be about 100 nm. Furthermore, specific core diameter measurements of exemplified ENPs described herein are provided in Table 1.

ENPs formed with a core may also include other structural features. For example, molecules may be associated with the core to facilitate particle dispersion in solution, influence overall particle charge or zeta potential, to target the ENP to a particular cell type, or to allow the particle to incorporate a cargo, such as a pharmacological agent. Polymeric conjugates provided herein may be useful to improve solubility and/or stability of a bioactive/therapeutic agent, reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues. Poorly soluble and/or toxic compounds may benefit particularly from incorporation into polymeric compounds of the invention. In some embodiments ore-associated molecules may be chemical polymers. In some embodiments a core particle may be associated, covalently or non-covalently, with a hydrophilic polymer. In some embodiments hydrophilic polymers associated with a core particle may be a polymer or copolymer (block or random) of poly(ethylene glycol), polyvinyl alcohol, polyvinyl acid, poly(meth)acrylate, poly(meth)acrylamide, polylactic acid (PLA), polyglycolic acid (PGA), polycaprolactone (PCL), poly(Lactide-co-Glycolide) (PLGA), collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polypeptides, proteins, polysaccharides, hyaluronic acid and alginate, acyl-substituted cellulose acetates, polyethylene oxide, glycerin, sorbitol, mannitol, sucrose, sorbitan, glycerol, xylitol, isomalt, polypropylene glycol, and poly(tetramethylene ether)glycol, polycaprolactones or polyester adipate polyols, polyether polyols, trehalose, lactose, glucose, or dextran, as well as biopolymers known in the art, may also be suitable for use in this regard.

The molecular weight of the described polymers may be from about 2,000 daltons to about 10,000 daltons. Nonetheless, those skilled in the art will understand that additional polymers and various polymer molecular weight combinations not expressly provided herein will allow for the production of ENPs that perform with substantial similarity to the ENPs described herein. In some embodiments the described polymers may have a molecular weight of about 2,000 daltons. In some embodiments the described polymers may have a molecular weight of about 3,000 daltons. In some embodiments the described polymers may have a molecular weight of about 4,000 daltons. In some embodiments the described polymers may have a molecular weight of about 5,000 daltons. In some embodiments the described polymers may have a molecular weight of about 6,000 daltons. In some embodiments the described polymers may have a molecular weight of about 7,000 daltons. In some embodiments the described polymers may have a molecular weight of about 8,000 daltons. In some embodiments the described polymers may have a molecular weight of about 9,000 daltons. In some embodiments the described polymers may have a molecular weight of about 10,000 daltons. In some embodiments the described ENPs include a core particle, such as a gold core particle, associated with poly(ethylene glycol). In some embodiments poly(ethylene glycol) may be covalently bound to a gold core particle as described herein. In some embodiments the described poly(ethylene glycol)polymers may have a molecular weight of about 2,000 daltons. In some embodiments the described poly(ethylene glycol)polymers may have a molecular weight of about 3,000 daltons. In some embodiments the described poly(ethylene glycol)polymers may have a molecular weight of about 4,000 daltons. In some embodiments the described poly(ethylene glycol)polymers may have a molecular weight of about 5,000 daltons. In some embodiments the described poly(ethylene glycol)polymers may have a molecular weight of about 6,000 daltons. In some embodiments the described poly(ethylene glycol)polymers may have a molecular weight of about 7,000 daltons. In some embodiments the described poly(ethylene glycol)polymers may have a molecular weight of about 8,000 daltons. In some embodiments the described poly(ethylene glycol)polymers may have a molecular weight of about 9,000 daltons. In some embodiments the described poly(ethylene glycol)polymers may have a molecular weight of about 10,000 daltons.

The described ENPs may also include a targeting moiety that facilitates localization to a particular part of the body or a certain cell type, increases the association with a particular cell, or fosters entry into a cell. The targeting moieties described herein may be a protein or a fragment thereof, a glycoprotein or a fragment thereof, a sugar, a starch, a chemical agent, a cytokine, a hormone, or a derivative thereof. In some embodiments the targeting agent may be a ligand for a receptor protein expressed by a cell. As will be appreciated by those in the art, the ENPs described herein can be applied locally or systemically administered (e.g., injected intravenously), thus, preferred targeting moieties are those that allow for concentration of the administered ENPs in a particular localization. In preferred embodiments, the targeting moiety allows targeting of the carrier particles of the invention to a particular tissue or the surface of a cell. In some embodiments the targeting moiety fosters uptake of the described ENPs by a cell.

In some embodiments, the targeting moiety is all or a portion (e.g., a binding portion) of a ligand for a cell surface receptor. Suitable ligands include, but are not limited to, peptides, hormones, lipids, proteins, glycoproteins, signal transducers, growth factors, cytokines, and others. The ligands may be human or derived from a human, or may be from any other animal, including cow, pig, sheep, dog, rabbit, rat, mouse, hamster, chicken, frog, monkey, or any other bovine, canine or avian species. The native sequences of suitable ligands are readily available in GenBank and other public databases, for example sequences of human transferrins are available in GenBank as Accession numbers NM001063, XM002793, XM039847, NM002343 and NM013900, among others. In some embodiments, the targeting moiety is an antibody. The term “antibody” includes entire antibodies as well as antibody fragments (such as Fv, Fab, Fab′, F(ab′) 2 or other antigen-binding subsequences of antibodies), and encompasses human antibodies, fully human antibodies such as those produced via phage display or transgenic mice having human immunoglobulin genes, humanized antibodies, chimeric antibodies, etc., either produced by the modification of whole antibodies or those synthesized de novo using recombinant DNA technologies. In a preferred embodiment, the antibody is directed against a receptor on the cell-surface, such as a transferrin receptor. In some embodiments, the targeting moiety is transferrin or a portion thereof that is capable of binding to a transferrin receptor. In some embodiments the targeting moiety is mannose, or a derivative thereof that is capable of binding to a mannose receptor.

The targeting moieties described herein may be associated with the ENP in a number of ways known in the art. For example, in some embodiments targeting moieties are attached to an ENP via linkage to a core associated polymer, such as poly(ethylene glycol). The carrier particles may include a targeting moiety to target the carrier particles (including therapeutic or diagnostic agents associated with the carrier particles) to a specific cell type, or a particular subcellular location. More than one targeting moiety can be conjugated or otherwise associated with a carrier particle, and the target molecule for each targeting moiety can be the same or different.

The hydrodynamic diameter of ENPs described herein may fall within the range of about 25 nm to about 125 nm. In some embodiments the ENPs may have a hydrodynamic diameter in the range of about 40 nm to about 100 nm. In some embodiments the ENPs may have a hydrodynamic diameter in the range of about 60 nm to about 100 nm. In some embodiments the ENPs may have a hydrodynamic diameter in the range of about 70 nm to about 90 nm. In some embodiments the hydrodynamic diameter of the described ENPs may be about 25 nm. In some embodiments provided herein the hydrodynamic diameter of the described ENPs may be about 30 nm. In some embodiments provided herein the hydrodynamic diameter of the described ENPs may be about 35 nm. In some embodiments the hydrodynamic diameter of the described ENPs may be about 40 nm. In some embodiments the hydrodynamic diameter of the described ENPs may be about 45 nm. In some embodiments provided herein the hydrodynamic diameter of the described ENPs may be about 50 nm. In some embodiments the hydrodynamic diameter of the described ENPs may be about 55 nm. In some embodiments the hydrodynamic diameter of the described ENPs may be about 60 nm. In some embodiments the hydrodynamic diameter of the described ENPs may be about 65 nm. In some embodiments provided herein the hydrodynamic diameter of the described ENPs may be about 70 nm. In some embodiments the hydrodynamic diameter of the described ENPs may be about 75 nm. In some embodiments the hydrodynamic diameter of the described ENPs may be about 80 nm. In some embodiments provided herein the hydrodynamic diameter of the described ENPs may be about 85 nm. In some embodiments the hydrodynamic diameter of the described ENPs may be about 90 nm. In some embodiments the hydrodynamic diameter of the described ENPs may be about 95 nm. In some embodiments the hydrodynamic diameter of the described ENPs may be about 100 nm. In some embodiments provided herein the hydrodynamic diameter of the described ENPs may be about 105 nm. In some embodiments the hydrodynamic diameter of the described ENPs may be about 110 nm. In some embodiments provided herein the hydrodynamic diameter of the described ENPs may be about 115 nm. In some embodiments the hydrodynamic diameter of the described ENPs may be about 120 nm. In some embodiments provided herein the hydrodynamic diameter of the described ENPs may be about 125 nm. Furthermore, specific hydrodynamic diameter measurements of exemplified ENPs described herein are provided in Table 1.

In some embodiments the described ENPs may exhibit a charge, providing for a cationic or anionic ENP. The degree of the charge may vary, depending on various characteristics of a given ENP. In other embodiments, the described ENPs may be neutral and have no charge. In this regard, the ENPs may have a zeta potential in either the positive or negative range. In some embodiments, the zeta potential may be from about 25 mV to about −25 mV. In other embodiments the zeta potential of the ENP may be from about −8 mV to about −14 mV. In some embodiments the ENP is a cationic particle. In some embodiments the ENP is an anionic particle. In some embodiments described herein the ENP is a particle having a gold core and a negative charge. For example, the zeta potential of such a particle may be in the range of −8 mV to −14 mV. In some embodiments the zeta potential of the described ENP may be −8 mV. In some embodiments the zeta potential of the described ENP may be −9 mV. In some embodiments the zeta potential of the described ENP may be −10 mV. In some embodiments the zeta potential of the described ENP may be −11 mV. In some embodiments the zeta potential of the described ENP may be −12 mV. In some embodiments the zeta potential of the described ENP may be −13 mV. In some embodiments the zeta potential of the described ENP may be −14 mV. For some ENPs exemplified herein, specific zeta potential values are provided in Table 1.

TABLE 1
Table 1: Physicochemical properties and in vivo characteristics of Aux-PEGy NPs.

| | Core(x) | PEG(y) | HD-water | | ZP | | Ω | | |
|---|---|---|---|---|---|---|---|---|---|
| NP | nm | Da | Nm | HD-1 × PBS nm | mV | t_{1/2} h | % ID | GTE % | SI |
| Au₅-PEG₅₀₀₀ | 5.3 ± 0.5 | 5000 | 26.2 ± 0.3 | 24.8 ± 0.5 | −8.44 ± 0.85 | 48.9 | 0.2 ± 0.1 | 0 | 0 |
| Au₂₀-PEG₅₀₀₀ | 21.6 ± 0.2 | 5000 | 43.1 ± 0.2 | 41.4 ± 0.2 | −9.62 ± 0.62 | 31.8 | 1.2 ± 0.5 | 50 | + |
| Au₄₀-PEG₄₀₀₀ | 41.2 ± 0.2 | 3910 | 59.1 ± 0.3 | 58.6 ± 0.5 | −12.34 ± 1.21 | 13.8 | 3.0 ± 0.6 | 80 | ++ |
| Au₅₀-PEG₅₀₀₀ | 51.4 ± 0.2 | 5000 | 78.8 ± 0.2 | 76.5 ± 0.4 | −10.91 ± 1.33 | 13.7 | 4.6 ± 0.9 | 100 | +++ |
| Au₆₀-PEG₇₀₀₀ | 58.1 ± 0.5 | 7000 | 94.6 ± 0.5 | 96.2 ± 0.2 | −12.51 ± 1.24 | 11.4 | 1.9 ± 0.4 | 90 | +++ |
| Au₈₀- | 76.5 ± 0.3 | 10000 | 127.6 ± 2.1 | 128.9 ± 0.9 | −8.93 ± 0.67 | 8.7 | 0.7 ± 0.4 | 70 | + |
| PEG₁₀₀₀₀ | | | | | | | | | |
| Au₁₀₀- | 98.3 ± 0.3 | 20000 | 167.4 ± 8.6 | 164.3 ± 8.6 | −9.76 ± 0.31 | 6.8 | 0.5 ± 0.3 | 60 | + |
| PEG₂₀₀₀ | | | | | | | | | |

x = core diameter of AuNP;
y = chain length of grafted PEG;
HD = hydrodynamic diameter;
ZP = ζ- potential in 1 mM KCl;
t_{1/2} = blood half-life;
Ω = kidney bulk particle content;
GTE = glomerular targeting efficiency;
SI = staining index (an arbitrary score that ranks both the intensity and spread of the silver stain, whereby +++ and 0 are the maximum and minimum values, respectively).
The table presents in vitro data as average ± s.d. from triplicates of experiments as well as in vivo data as average ± s.d. from three animals per particle type.

The ENPs described herein may be associated with a therapeutic agent. In some embodiments the therapeutic agent may be a polynucleotide, a protein or protein fragment, a radionuclide, or a pharmaceutical agent. In some embodiments the polynucleotide associated with the described ENPs is capable of inhibiting protein production, such as an inhibitory RNA polynucleotide. In the instance of ENPs having a gold, or gold-based, core, it is well known in the art that polynucleotides, such as siRNA, may be conjugated directly to the core particle via gold-thiol interactions (Lytton-Jean, et al., Small 7(14):1932 (2011)). This method, though described and exemplified herein should not be considered to exclude the use of alternative methods which also allow for the addition of therapeutic agents, such as siRNA, to ENPs. For example, one such alternative is layer-by-layer fabrication, which makes use of charged polymers to add multiple layers of thin films onto the surface of a particle (Lee, et al., Small 7(3):364 (2011)).

Also provided is a diagnostic method for administering to a subject an ENP, analyzing a mesangial cell of the subject and determining whether the engineered nanoparticle is present in a mesangial cell of the subject. In this regard, the described nanoparticles may include a detectable agent, such as an epitope tag, a radiolabel, a fluorophore, a polynucleotide encoding a protein of interest, or a polynucleotide capable of preventing the expression of a protein of interest. In some embodiments the ENP may include an inhibitory RNA polynucleotide capable of preventing the expression of a protein of interest, which allows for identification of cells in which the described ENP is present. For example, in one embodiment a cell expressing green fluorescent protein (GFP) may be identified to have internalized an ENP carrying an inhibitory RNA specific for GFP. In some embodiments the ENP may contact a mesangial cell of a subject as a fully intact particle, while in other embodiments the ENP may enter a mesangial cell of a subject as a fully intact particle. The described methods of detection may be performed by analyzing a subject, or a biological sample obtained from a subject. Methods for carrying out an analysis of this nature are known to those skilled in the art. Some examples of such methods include electron microscopy, fluorescence microscopy or computed axial tomography (i.e., CAT scan).

### EXAMPLES

The subject matter provided herein may be understood more readily by reference to the following detailed description taken in connection with the accompanying figures and examples, which form a part of this disclosure.

### Example I

### Size-dependent Nanoparticle Accumulation in the Kidney

Studies were undertaken to assess the ability of nanoparticle size to influence particle accumulation in various organs. Gold-based nanoparticles (AuNPs), a type of ENP, were selected for these studies, as they are compatible with multiple imaging methods. As rigid and non-decomposable objects, they cannot escape the kidney by renal clearance. Unmodified gold nanoparticles of different sizes have zeta potentials (ζ) ranging from −19 mV to −28 mV. Measured ζ values were consistent with predictions due to classical electrokinetic theory (FIG. 2), and suggest that unmodified AuNPs of all sizes share the same surface charge density (σ).

For any charged sphere of radius R in an electrolyte, its zeta potential (ζ) is given as follows:

$$ξ = \frac{σ}{ɛ} \frac{R}{1 + kR}$$

Above π, ε, and k⁻¹ are the surface charge density, permittivity constant, and Debye length. From this equation, ζ of charged gold spheres becomes more negative as R increases, consistent with data shown in Table I. For typical measurements in 1 mM KCl at room temperature, k⁻¹ is roughly 9.8 nm, a constant independent of R. By curve fitting of the measured ζ as a function of R, the dimensionless charge density (σ/ε) is approximately 3. This means that unmodified gold surface of all sizes have a constant surface charge density.

Poly(ethylene glycol) (PEG) was used to create particles having the same surface charge, given the charge screening effect associated with PEG. Larger gold particels, which have a more negative surface charge, required the engraftment of longer PEG chains onto their surface to provide sufficient charge screening. To more fully assess the interplay between AuNP size and PEG length, an assortment of PEGylated gold nanoparticles (Auₓ-PEG_{y} NPs) using gold particles of different core diameters (x) and PEG of different chain lengths (y) were produced (Table 1). The engraftment procedure entailed the use of methoxy-PEG-thiol (mPEG-SH), whose terminal thiol group can react with the gold surface via the formation of gold-thiol covalent bonds in deionized water. Careful choice of core diameter and PEG chain length gave rise to a near-constant ζ value (roughly−10 mV) for Auₓ-PEG_{y} NPs of various final hydrodynamic sizes (Table 1). In general, the engraftment of each additional 2000 molecular weight of PEG onto the gold surface translates to an increase in 5 nm of the hydrodynamic diameter of Aux-PEG_{y} NPs. This linearity between PEG corona thickness and chain length is consistent with previous predictions for tethered polymer brushes on spherical interfaces without pronounced curvature (Tables 1 and 2). All Auₓ-PEG_{y} NPs showed remarkable stability in salt after 24 h, with hydrodynamic sizes in phosphate-buffered saline (PBS) roughly equal to those in water. Dan, N. and Tirrell, M., Macromolecules 25, 2890-2895 (1992).

To assess accumulation of Auₓ-PEG_{y} NPs, balb/c mice (N=3) received single intravenous injections of each type of Auₓ-PEG_{y} NPs at the same particle concentration. From each mouse, blood was withdrawn via the saphenous vein at various time points to evaluate its gold content using inductively coupled plasma mass spectrometry (ICP-MS). With extensive surface engraftment of PEG (y≧4000), all particles manifested extended blood circulation with a half-life (t_{1/2}) spanning from 7 h to 38 h (Table 1). Particle size and half-life were inversely correlated (FIG. 3A). The simultaneous increase in x and y led to reduction in half-life, indicating that size-dependent internal clearance, not colloidal stability conferred by PEGylation, played a more dominant role in determining particle blood circulation.

Mice were sacrificed 24 h after injection to extract organs for detection of bulk gold content using ICP-MS. For all particle sizes, gold content of the six organs plus the blood samples collected at three time points summed up to ≧70% injected dose (ID), thus constituting a mass balance that accounted for the destinations of most injected Auₓ-PEG_{y} NPs. Overall, the liver, spleen, and kidney were the chief sites of particle accumulation, whereas the lung, pancreas, and heart showed negligible (<0.5% ID) particle retention (FIG. 3B). The liver and spleen both showed a positive correlation between particle size and degree of particle uptake, in agreement with previous reports that the degree of particle phagocytosis by Kupffer cells and spleen macrophages is largely size-dependent (Sadauskas, E. et al., Part. Fibre. Toxicol. 4, 1-7 (2007)). Approximately 50-60% ID of the larger particles (Au₆₀-PEG₇₀₀₀, Au₅₀-PEG₁₀₀₀₀, Au₁₀₀-PEG₂₀₀₀₀) resided in organs associated with the reticuloendothelial system (RES). In accord to the mass balance, this RES uptake explained their lowest retention in blood circulation (5-20% ID). Conversely, the blood particle content of the smallest particles (Au₅-PEG₅₀₀₀ and Au₂₀-PEG₅₀₀₀) was the most pronounced (43% ID). Unexpectedly, particles were observed to accumulate in the kidney in a size-dependent manner. Despite that a lower fraction of the injected dose accumulated in the kidney, relative to the liver and spleen (1-5% ID), peak retention was observed for Au₅₀-PEG₅₀₀₀ particles (FIG. 4).

### Example II

### Localization of Auₓ-PEG_{y} NPs within the Kidney

To understand the size-dependent accumulation observed in the kidney, “silver enhanced” kidney sections were prepared to examine the distribution of Auₓ-PEG_{y} NPs at the tissue level. Gold selectively catalyzes the reduction of silver ions and deposition of metallic silver, making nanosized objects embedded in kidney sections visible under light microscopy. Within the cortex, most particles resided either within the lining of the peritubular capillaries (likely in resident macrophages), which intertwine the cortical tubules (proximal convoluted tubules and distal convoluted tubules), or inside renal corpuscles. Particle accumulation nearby peritubular capillaries was independent of size, because silver intensity and scattering did not show clear correlation with size (FIG. 5). However, particle accumulation inside renal corpuscles is a strong function of size (FIG. 4). The smallest particles (Au₅-PEG₅₀₀₀) were virtually undetectable in the renal corpuscles, but were found in peritubular capillaries (FIG. 4A). The next smallest particles (Au₂₀-PEG₅₀₀₀) merely accumulated in the renal corpuscles and were rarely observed in the lining of the peritubular capillaries. Only about 50% of the renal corpuscles contained Au₂₀-PEG₅₀₀₀ NPs (FIG. 4B). The staining intensity appeared to be mild. For Au₄₀-PEG₄₀₀₀ NPs, particle staining within the renal corpuscles became more intense (FIG. 4C), in which about 80% of the renal corpuscles were stained positive for particles. Similar accumulation patterns were apparent for Au₅₀-PEG₅₀₀₀ NPs, except that 100% of the renal corpuscles examined under the light microscope stained positive for these particles (FIG. 4D). Incidentally, this perfect glomerular targeting efficiency (GTE) matches strongly with the maximal bulk particle content in the kidney observed for Au₅₀-PEG₅₀₀₀ NPS. Closer inspection of these renal corpuscles also reveals the most intense silver throughout the largest area fraction of the renal corpuscles. The GTE for Au₆₀-PEG₇₀₀₀ NPs was roughly 90%, and such particles also elicited very intense silver staining at more spatially confined regions of the renal corpuscles (FIG. 4E). Au₈₀-PEG₁₀₀₀₀ and Au₁₀₀-PEG₂₀₀₀₀ NPs recorded a GTE of 60-70%.

Due to the catalytic nature of staining, larger Aux-PEGy NPs are expected to receive more silver deposition on their periphery. While silver staining can confirm the presence of Auₓ-PEG_{y} NPs, its intensity alone does not warrant the quantitation of actual particle content. Thus, besides the absolute magnitude of intensity, the spread of staining (areal fraction covered by silver) within renal corpuscles is also an important measure. A different approach to understanding the nanoparticle distribution in the kidney is to determine the “staining index (SI),” an arbitrary measure that accounts for both the intensity and spread of staining (SI listed in Table 3). For the largest nanoparticles, silver staining was found in more limited regions of renal corpuscles (FIG. 4F-G), despite their higher staining. Overall, the staining index of the largest nanoparticles (Au₈₀-PEG₁₀₀₀₀ and Au₁₀₀-PEG₂₀₀₀₀ NPs) was lower than that of Au₈₀-PEG₁₀₀₀₀ NPs.

Based on a previous work by Takae et al, each 20 nm AuNP can anchor 520 PEG chains of 6000 Da each. This translates to a PEG grafting density (σ*) of 0.4 PEG/nm2 The Kuhn length (b) of PEG is 0.7 nm. Thus the dimensionless PEG grafting density (σ*) is: σ*=b²Σ=(0.7 nm)² (0.4 PEG/nm²)=0.196.

Hill et al. evaluated the grafting density of oligonucleotides on submicron sized AuNPs (10-100 nm). If the oligonucleotide density data due to size curvature is applicable to the study of PEG grafting, then a rough estimate of PEG grafting density of Aux-PEGy NPs is provided below:

TABLE 2
Rough estimates of PEG grafting density on
AuNPs of different sizes.

| Core | Hil et al | | |
|---|---|---|---|
| size (x) | oligo | PEG | |
| (nm) | (#oligo/nm²) | (#PEG/nm²) | PEG - σ* |
| 20 | 1.40E+13 | 4.00E−01 | 0.196 |
| 40 | 8.50E+12 | 2.43E−01 | 0.119 |
| 50 | 8.10E+12 | 2.31E−01 | 0.1134 |
| 60 | 7.80E+12 | 2.23E−01 | 0.1092 |
| 80 | 7.10E+12 | 2.03E−01 | 0.0994 |
| 100 | 7.10E+12 | 2.03E−01 | 0.0994 |

σ* takes on the value of 0.1-0.2 (for AuNPs above the size of 20 nm), which represents a very high grafting density according to Wijman et al. Physically, how high is this density?

According to scaling analysis by deGennes on grafted polymers, the brush conformation appears if o*>N-6/5, where N is the number of Kuhn polymer segments. How can we estimate N?

Take Au50-PEG5000 as an example. The MW of each PEG unit is 44 g/mol. The two C—O bonds (each 0.145 nm) and C—C bond (0.15 nm) add up to 0.44 nm. The contour length (Rmax=Nb) of a fully stretched PEG5000 coil is 5000/44*0.44 nm=50 nm. If b=0.7 nm, then N=71.4. Clearly, o* is greater than N-6/5. Hence, PEG polymer chains fill are overlapping with each other, with their blobs acting as hard spheres and cover the gold surface densely. The o*>N-6/5 result is also apparent for all other Aux-PEGy NPs.

Alternatively, one can calculate the footprint (D) of each PEG chains (separation distance between each adjacent PEG chain) on the gold surface, knowing that 4πD²=Σ. For Au50-PEG5000, D=2.29 nm, which is shorter than the Flory radius of PEG5000 in a good solvent (RF=bN3/5=9.07 nm). Because RF>D result is also apparent for all other Aux-PEGy NPs, the grafted PEG chains all take the “brush conformation” on the gold surface for all particle sizes.

TABLE 3
Polymer parameters of grafted PEG on AuNPs.

| | | | | | |
|---|---|---|---|---|---|
| MW (g/mol) | 4000 | 5000 | 7000 | 10000 | 20000 |
| R_max (nm) | 40 | 50 | 70 | 100 | 200 |
| N | 57.14286 | 71.42857 | 100 | 142.8571 | 285.7143 |
| N-6/5 | 0.007792 | 0.005961 | 0.003981 | 0.002595 | 0.001129 |
| R_F (nm) | 7.930037 | 9.066115 | 11.09425 | 13.74166 | 20.82846 |
| D (nm) | 2.289032 | 2.347735 | 2.389476 | 2.504419 | 2.504419 |
| x (nm) | 40 | 50 | 60 | 80 | 100 |
| x/2b | 28.57143 | 35.71429 | 42.85714 | 57.14286 | 71.42857 |

MW: molecular weight;
Rmax = bN: contour length;
N: degree of polymerization (no. of Kuhn segments);
RF = bN3/5: Flory radius in a good solvent;
D: separation distance between PEG monomers;
x = core size of AuNP.

### Example III

### Intracellular Localization of Auₓ-PEG_{y} NPs in the Renal Cortex

Upon confirmation of particle glomerular targeting, transmission electron microscopy (TEM) was used to delineate particle intracellular localization patterns in the renal cortex. These efforts were focused on Auₓ-PEG_{y} NPs of intermediate sizes that yielded the higher overall particle content in the bulk kidney and GTE. In agreement with histological data, particles of all sizes were either engulfed by endothelial cells or resident macrophages or remained as isolated entities in circulation inside pertitubular blood capillaries. Particle accumulation in peritubular blood capillaries was not size-dependent (FIG. 6). Retention of particles in renal corpuscles, however, was a strong function of size (FIG. 7). Smaller particles (Au₂₀-PEG₅₀₀₀ NPs) entered the mesangium within renal corpuscles in minute quantities (FIG. 7A). As size increases, Auₓ-PEG_{y} NPs showed more association with mesangial cells. Au₄₀-PEG₄₀₀₀, Au₅₀-PEG₅₀₀₀, and Au₆₀-PEG₇₀₀₀ NPs were found in multiple clusters distributed throughout the mesangium. Particles were either within mesangial cells or in the extracellular matrix outside mesangial cells (FIG. 7B-D). Larger particles (Au₅₀-PEG₁₀₀₀₀ NPs) only resided at isolated amounts (FIG. 7E). Au₅₀-PEG₅₀₀₀ NPs were observed to be associated with mesangial cells more than any other particle, which is consistent with the bulk kidney particle content and glomerular targeting efficiency results provided herein. Thus, Au₅₀-PEG₅₀₀₀ represents the particle size that maximizes kidney targeting at organ, tissue, and cellular (mesangium) levels.

Further analysis of the TEM data suggests that nanoparticle surface charge may also play a role in glomerular deposition of Auₓ-PEG_{y}NPS. Nanoparticles of <100 nm in at least 2 dimensions should be able to pass through the glomerular endothelial fenestrations and gain access to the mesangium and basement membrane (Bennett, K. M. et al., Magnetic Resonance in Medicine, 60(3):564-74 (2008); Ruggiero, A. et al., Proc. Natl. Acad. Sci. USA. 107(27):12369-12374 (2010)). Despite this fact, Auₓ-PEG_{y}NPS were not observed in the glomerular basement membrane. Given that the GBM is a highly negatively charged space, it is possible that the slight negative surface potential of the Auₓ-PEG_{y}NPS (˜−10 mV) disfavors entry into this structure. Bennet et al., clearly demonstrate that positively charged ferritin nanoparticles do accumulate in the GBM following i.v. administration in mice. Therefore, it may be that modulating the surface charge of the Aux-PEG_{y}NPS results in changes to glomerular deposition of the particles. Specifically, gold particles possessing positive surface potentials should accumulate in the GBM in addition to the mesagium resulting in an increase in overall number of Auₓ-PEG_{y}NPS deposited within the glomerulus.

This provides a better understanding of in vivo behavior of submicron sized nanoparticles in the kidney as a sole function of size in the 10-150 nm size range. From blood pharmacokinetics as well as in vivo distribution patterns at the organ, tissue, and cellular levels, the provided results suggests a particle size (Au₅₀-PEG₅₀₀₀NPs) that maximizes bulk particle uptake in the kidney, deposition of particles in renal corpuscles within the cortex, and uptake of particles by mesangial cells within renal corpuscles. When using nanoparticles as a cancer therapeutic, even their accumulation in single digit % ID amounts in the tumor can lead to gene inhibition and tumor reduction. (Bartlett, et al., Proc. Natl. Acad. Sci. USA. 104, 15549-15554 (2007)). Thus, the accumulation of Aux-PEGy NPs (1-5% ID) in the kidney is likely to impart clinical effects to target kidney diseases.

### Materials and Methods

The following materials and methods were used in the experiments described in the foregoing examples.

General: Unless otherwise mentioned, all poly(ethylene glycol) (PEG) raw materials were purchased from Laysan Bio. All organic solvents were purchased from Sigma. Phosphatebuffered saline (PBS) comprises 150 mM NaCl and 50 mM sodium phosphate (pH=7.4).

Synthesis of mPEG₄₀₀₀-SH and mPEG₇₀₀₀-SH: 50 mg of amine-PEG₃₄₀₀-thiol (14.7 μmol) was reacted with 40.4 mg of methoxy-PEG₅₅₀-(succinimidyl propionate) (73.5 μmol) in 50 μL of triethylamine (TEA) and 1.2 mL of anhydrous dichloromethane (DCM). The reaction proceeded at room temperature (RT) with stirring for 7 h. The crude mixture was dried under vacuuo, and dialyzed against deionized water using a 3 kDa Amicon MWCO membrane (Millipore) for three times. Likewise, 15 mg of amine-PEG₅₀₀₀-thiol (3.0 μmol) was reacted with 60 mg of methoxy-PEG₂₀₀₀-(succinimidyl valerate) (30.0 μmol) in 50 μL of TEA and 1.2 mL of anhydrous DCM. The reaction proceeded at RT with stirring for 9 h. The crude mixture was dried under vacuuo, and dialyzed against deionized water using a 20 kDa Amicon MWCO membrane (Millipore) for three times. The correct fraction (7000 Da) was separated using HPLC using a size exclusion column. Final molecular weights were confirmed by MALDI-TOF.

Assembly of PEGylated gold nanoparticles (Auₓ-PEG_{y} NPs): Methoxy-PEG-thiol (purchased or synthesized above) of a certain molecular weight (y=4000, 5000, 7000, 10000, and 20000), dissolved in deionized water was added to 3 mL of aqueous suspension of unconjugated gold colloids (Ted Pella) of a designated core size (x=5 nm, 20 nm, 40 nm, 50 nm, 60 nm, 80 nm, and 100 nm) at an excess concentration of ˜9 PEG strands per nm² of gold surface. e. g. To ensure complete coverage, PEGylation of 50 nm AuNPs required the addition of 10 μL of 1 mM mPEG5000-thiol (in deionized water) to 0.5 mL of aqueous suspension of 2.25×10¹⁰ particles. All PEGylation reactions proceeded at room temperature for 30 min with stirring. To remove any unbound methoxy-PEG-thiol, the reaction mixture was dialyzed against deionized water using a 50 kDa Amicon MWCO membrane (Millipore) for three times.

Physicochemical characterization of Auₓ-PEG_{y} NPs: Hydrodynamic diameter (HD) and ζ-potential (ZP) of Auₓ-PEG_{y} NPs were measured using ZetaPals (Brookhaven). For HD measurements, the particle pellet was re-suspended in 1.2 mL of deionized water or PBS. Reported HDs are average values from 3 runs of 3 minutes each. For ZP analysis, the pellet was re-suspended in 1.4 mL of 1 mM KCl. Reported ZPs are average values from 10 runs each with a target residual of 0.012 measured at a conductance of 320±32 μS.

Animal experiments: For each type of Auₓ-PEG_{y} NPs, three 9-week, female Balb/c mice (Jackson Laboratory) received i.v. injections of particles via the tail vein at a concentration of 4.5 10¹¹ particles per mL, formulated in 120 μL of filtered 5% dextrose in deionized water (D5W). At three consecutive time points after injection (30 min, 4 h, and 24 h), 30 μL of mouse blood was drawn from each mouse via its saphenous vein using Microvette CB 300 Capillary Blood Collection Tube with EDTA (Sarstedt). Blood samples were stored at 4° C. for future use. After 24 h, mice were euthanized by CO₂ overdose for the collection of the liver, kidney, lung, heart, spleen, and pancreas. All organs were fixed in 4% paraformaldehyde (PFA) in PBS for 3 days.

ICP-MS: Homogenized organs were oxidized in 0.5 mL of acid mixture (70% HNO₃ and 35% HCl at a 3:1 volume ratio) in a microwave until they fully dissolved. After adding 20.5 mL of deionized water, the sample was centrifuged at 3200×g for 15 minutes to remove cell debris, leaving the supernatant for gold content analysis using HP 4500 ICP-MS (Agilent, Foster City, Calif.). Nebulization occurred with a flow of 1.3 L/min of argon using a Babbington type nebulizer in a pyrex Scott-type spray chamber. The argon plasma power was 1200 W with a flow of 15 L/min and an auxiliary flow of 1.1 L/min. A calibration curve against known concentrations of Auₓ-PEG_{y} NPs of all sizes was used to measure the gold content, using 2.5% HNO₃ and 0.42% HCl as the blank solvent and tissues from uninjected Balb/c mice to account for background organ gold content. Reported values are expressed as % of injected dose (% ID). Error bars indicate one s.d. in each mouse group (N=3). Each mouse weighed ˜20 g at the time of experiment, and had a total blood volume of 1.6 mL (average mouse volume is 77-80 μL/g).

Histology with silver enhancement: PFA-fixed organs were dehydrated and embedded in molten paraffin to generate sections of 4 μm thick. Deparrifinized sections were rehydrated with a reducing ethanol gradient and rinsed with deionized water extensively, dried, and stained for Auₓ-PEG_{y} NPs using the Silver Enhancement Kit for Light and Electron Microscopy (Ted Pella) in the dark for 25 min at RT. After rinsing with running tap water for 2 min, sections were counter-stained with Gill's 3 hematoxylin and 1% eosin (in 95% ethanol) for 40 s each, and then mounted with Permount for viewing under an Axioplan 2 light microscope (Zeiss) with a 40× objective. To estimate the targeting efficiency of particles to renal corpuscles from light micrographs, 300 renal corpuscles per injected mouse on random positions of kidney sections were inspected visually for the presence of silver stains.

TEM: Tissue blocks (˜1 mm³) were fixed in 2.5% glutaraldehyde (in 0.1M sodium cacodylate, pH=7.4) for 2 hours, stained by 1% OsO₄ at 4° C. for 2 hours, and 0.9% OsO₄ and 0.3% K₃Fe(CN)₆ at 4° C. for 2 hours. Gradual dehydration with ethanol and propylene oxide enabled tissue embedding in Epon 812 resins (Electron Microscopy Sciences, Hatfield, Pa.). 80 nm thick sections were deposited on carbon and formvar-coated, 200-mesh, nickel grids (EMS) and stained with 3% uranyl acetate and Reynolds lead citrate for visualization under either a 120 kV BioTwin CM120 TEM (Philips) or 300 kV TF30UT transmission electron microscope (FEI).

What is claimed:

1. A method of treating a disorder affecting the mesangial cells in a subject in need thereof comprising administering an engineered nanoparticle comprising a therapeutic agent to said subject.

2. The method of claim 1, said engineered nanoparticle having a hydrodynamic diameter in the range of about 55 nm to about 125 nm.

3. The method of claim 1, said engineered nanoparticle having a net negative charge.

4. The method of claim 1, said engineered nanoparticle comprising a hydrophilic polymer.

5. The method of claim 4, said hydrophilic polymer being a being poly(ethylene glycol), polyvinyl alcohol, polyvinyl acid, poly(meth)acrylate, poly(meth)acrylamide, polylactic acid (PLA), polyglycolic acid (PGA), polycaprolactone (PCL), poly(Lactide-co-Glycolide) (PLGA), collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polypeptides, proteins, polysaccharides, hyaluronic acid and alginate, acyl-substituted cellulose acetates, polyethylene oxide, glycerin, sorbitol, mannitol, sucrose, sorbitan, glycerol, xylitol, isomalt, polypropylene glycol, and poly(tetramethylene ether)glycol, polycaprolactones or polyester adipate polyols, polyether polyols, trehalose, lactose, glucose, or dextran.

6. The method of claim 1, said engineered nanoparticle comprising a core.

7. The method of claim 6, said engineered nanoparticle further comprising a molecule attached to the core.

8. The method of claim 7, said molecule comprising a hydrophilic polymer.

9. The method of claim 8, said hydrophilic polymer being a being poly(ethylene glycol), polyvinyl alcohol, polyvinyl acid, poly(meth)acrylate, poly(meth)acrylamide, polylactic acid (PLA), polyglycolic acid (PGA), polycaprolactone (PCL), poly(Lactide-co-Glycolide) (PLGA), collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polypeptides, proteins, polysaccharides, hyaluronic acid and alginate, acyl-substituted cellulose acetates, polyethylene oxide, glycerin, sorbitol, mannitol, sucrose, sorbitan, glycerol, xylitol, isomalt, polypropylene glycol, and poly(tetramethylene ether)glycol, polycaprolactones or polyester adipate polyols, polyether polyols, trehalose, lactose, glucose, or dextran.

10. The method of claim 8, said hydrophilic polymer being poly(ethylene glycol).

11. The method of claim 10, said poly(ethylene glycol)polymer having a molecular weight in the range of about 4,000 Da to about 10,000 Da.

12. The method of claim 10, said poly(ethylene glycol)polymer having a molecular weight in the range of about 4,000 Da to about 7,000 Da.

13. The method of claim 10, said poly(ethylene glycol)polymer having a molecular weight is about 5,000 Da.

14. The method of claim 8, said hydrophilic polymer further comprising a targeting moiety.

15. The method of claim 14, said targeting moiety being a protein or protein fragment.

16. The method of claim 14, said targeting moiety being transferrin or mannose.

17. The method of claim 6, said core comprising gold, iron(III) oxide, carbon, carbon nanotubes, cadmium selenide, titanium, titanium dioxide, tin, tin oxide, silicon, silicon dioxide, iron, nickel, silver, copper, aluminum, steel, titanium alloy, brushite, tricalcium phosphate, chitosan, alumina, silica, lipinds, polystyrene, polylactides, silicone rubber, polycarbonate, polyurethane, polypropylene, polymethylmethaacrylate, polyvinyl chloride, polyester, polyether, or polyethylene.

18. The method of claim 6, wherein the core is gold.

19. The method of claim 6, said core having a diameter of from about 40 nm to about 75 nm.

20. The method of claim 3, said negatively charged nanoparticle exhibiting a zeta potential of from about −8 mV to about −14 mV.

21. The method of claim 1, said subject being a mammal.

22. The method of claim 1, said subject being a human.

23. The method of claim 1, said therapeutic agent being a polynucleotide, a protein or protein fragment, a radionuclide, or a pharmaceutical agent.

24. The method of claim 23, said polynucleotide being an inhibitor RNA.

25. The method of claim 1, said engineered nanoparticle contacts a mesangial cell of the subject as an intact particle.

26. The method of claim 1, said engineered nanoparticle further comprising a targeting moiety.

27. The method of claim 26, said targeting moiety being a protein or protein fragment.

28. The method of claim 27, said targeting moiety being transferrin or mannose.

29. The method of claim 1, said therapeutic effect being initiated inside the mesangial cell of the subject.

30. The method of claim 1, said disorder affecting the mesangial cells being IgA nephropathy, lupus nephritis, diabetic nephropathy, focal and segmental glomeruluosclerosis, focal segmental glomeruluosclerosis, membranous nephropathy, membranoproliferative glomerulonephritis, or amyloidosis.

31. The method of claim 1, said engineered nanoparticle being administered systemically.

32. A diagnostic method comprising:
a. administering to a subject an engineered nanoparticle,
b. analyzing a mesangial cell of the subject; and
c. determining whether the engineered nanoparticle is present in a mesangial cell of the subject.

33. The method of claim 32, the engineered nanoparticle having a hydrodynamic diameter in the range of about 55 nm to about 125 nm.

34. The method of claim 32, said engineered nanoparticle having a net negative charge.

35. The method of claim 32, said engineered nanoparticle comprising a hydrophilic polymer.

36. The method of claim 36, said hydrophilic polymer being poly(ethylene glycol), polyvinyl alcohol, polyvinyl acid, poly(meth)acrylate, poly(meth)acrylamide, polylactic acid (PLA), polyglycolic acid (PGA), polycaprolactone (PCL), poly(Lactide-co-Glycolide) (PLGA), collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polypeptides, proteins, polysaccharides, hyaluronic acid and alginate, acyl-substituted cellulose acetates, polyethylene oxide, glycerin, sorbitol, mannitol, sucrose, sorbitan, glycerol, xylitol, isomalt, polypropylene glycol, and poly(tetramethylene ether)glycol, polycaprolactones or polyester adipate polyols, polyether polyols, trehalose, lactose, glucose, or dextran.

37. The method of claim 32, said engineered nanoparticle comprising a core.

38. The method of claim 37, said engineered nanoparticle further comprising a molecule attached to the core.

39. The method of claim 38, said molecule comprising a hydrophilic polymer.

40. The method of claim 39, said hydrophilic polymer comprising a being poly(ethylene glycol), polyvinyl alcohol, polyvinyl acid, poly(meth)acrylate, poly(meth)acrylamide, polylactic acid (PLA), polyglycolic acid (PGA), polycaprolactone (PCL), poly(Lactide-co-Glycolide) (PLGA), collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polypeptides, proteins, polysaccharides, hyaluronic acid and alginate, acyl-substituted cellulose acetates, polyethylene oxide, glycerin, sorbitol, mannitol, sucrose, sorbitan, glycerol, xylitol, isomalt, polypropylene glycol, and poly(tetramethylene ether)glycol, polycaprolactones or polyester adipate polyols, polyether polyols, trehalose, lactose, glucose, or dextran.

41. The method of claim 39, said hydrophilic polymer comprising poly(ethylene glycol).

42. The method of claim 41, wherein the molecular weight of poly(ethylene glycol) is in the range of about 4,000 Da to about 10,000 Da.

43. The method of claim 41, wherein the molecular weight of poly(ethylene glycol) is in the range of about 4,000 Da to about 7,000 Da.

44. The method of claim 41, wherein the molecular weight of poly(ethylene glycol) is about 5,000 Da.

45. The method of claim 39, said hydrophilic polymer further comprising a targeting moiety.

46. The method of claim 45, said targeting moiety being a protein or protein fragment.

47. The method of claim 45, said targeting moiety being transferrin or mannose.

48. The method of claim 37, said core comprising gold, iron(III) oxide, carbon, carbon nanotubes, cadmium selenide, titanium, titanium dioxide, tin, tin oxide, silicon, silicon dioxide, iron, nickel, silver, copper, aluminum, steel, titanium alloy, brushite, tricalcium phosphate, chitosan, alumina, silica, lipinds, polystyrene, polylactides, silicone rubber, polycarbonate, polyurethane, polypropylene, polymethylmethaacrylate, polyvinyl chloride, polyester, polyether, or polyethylene.

49. The method of claim 37, wherein the core is gold.

50. The method of claim 37, said core having a diameter of from about 40 nm to about 75 nm.

51. The method of claim 34, said negatively charged nanoparticle exhibiting a zeta potential of from about −8 mV to about −14 mV.

52. The method of claim 32, said subject being a mammal.

53. The method of claim 32, said subject being a human.

54. The method of claim 32, said engineered nanoparticle further comprising a detectable agent.

55. The method of claim 54, said detectable agent being an epitope tag, a radiolabel, a polynucleotide encoding a protein of interest, or a polynucleotide capable of preventing the expression of a protein of interest.

56. The method of claim 55, said polynucleotide capable of preventing the expression of a protein of interest encoding an inhibitory RNA.

57. The method of claim 32, said engineered nanoparticle detected in a mesangial cell of the subject being intact.

58. The method of claim 32, wherein the mesangial cell of the subject is analyzed by electron microscopy, fluorescence microscopy or computed axial tomography.

59. The method of claim 32, said engineered nanoparticle further comprising a targeting moiety.

60. The method of claim 59, said targeting moiety being a protein or protein fragment.

61. The method of claim 60, said targeting moiety being transferrin or mannose.
